(19) United States
(12) Patent Application Publication
(10) Pub. No.: US 2014/0296178 A1
(43) Pub. Date: Oct. 2, 2014

(54) Combination Therapy with a Gallium Complex

(71) Applicant: Hooshmand Sheshbaradaran, Matawan, NJ (US)

(72) Inventor: Hooshmand Sheshbaradaran, Matawan, NJ (US)

(73) Assignee: Niiki Pharma Acquisition Corp. 2, Tampa, FL (US)

(21) Appl. No.: 13/991,356

(22) PCT Filed: Dec. 1, 2011
(86) PCT No.: PCT/US2011/062747

§ 371 Date: Sep. 12, 2013

Related U.S. Application Data
(60) Provisional application No. 61/418,840, filed on Dec. 1, 2010.

(51) Int. Cl. A61K 31/555; A61K 31/513; A61K 31/517; A61K 31/4188; A61K 31/436; A61K 31/454; A61K 31/337; A61K 31/7068

(52) U.S. Cl. CPC A61K 31/555; A61K 31/337; A61K 31/513; A61K 31/7068; A61K 31/4188; A61K 31/436; A61K 31/454; A61K 31/517
USPC 514/49; 514/187

(57) ABSTRACT

A combination therapy is disclosed for treating cancer. The method comprises identifying a patient diagnosed of cancer and in need of treatment, administering to a cancer patient a therapeutically effective amount of a compound of Formula (I), and administering to said patient a therapeutically effective amount of a certain second anti-cancer drug.

### CROSS-REFERENCE TO RELATED U.S. APPLICATIONS

This application claims the benefit of U.S. Provisional Application No. 61/418,840 filed on Dec. 1, 2010, the content of which is incorporated herein by reference.

### FIELD OF THE INVENTION

The present invention generally relates to pharmaceutical compositions and methods for treating cancer, and particularly to a combination therapy with a gallium complex.

### BACKGROUND OF THE INVENTION

Tris(8-quinolinolato)gallium(III) is an organic gallium complex that has been suggested to be useful in certain types of cancer. For example, U.S. Pat. No. 7,919,486 discloses and claims the use of tris(8-quinolinolato)gallium(III) and related compounds for the treatment of melanoma.

### SUMMARY OF THE INVENTION

It has been surprisingly discovered that the combined use of a compound of Formula (I) below, particularly tris(8-quinolinolato)gallium(III), and certain anti-cancer drugs can create unexpected synergies in killing selected tumor cells and therefore treating selected cancers. In a first aspect, the present invention provides a method of treating breast or colorectal cancer in a patient in need of such treatment comprising administering to the patient a therapeutically effective amount of a compound of Formula (I) and 5-fluorouracil or a prodrug thereof.

In a second aspect, the present invention provides a method of treating breast cancer in a patient in need of such treatment comprising administering to the patient a therapeutically effective amount of a compound of Formula (I) and paclitaxel or a prodrug thereof.

In a third aspect, the present invention provides a method of treating cancer (e.g., lung cancer or renal cell carcinoma) in a patient in need of such treatment comprising administering to the patient a therapeutically effective amount of a compound of Formula (I) and a second drug that is gemcitabine or temsirolimus.

In a fourth aspect, the present invention provides a method of treating prostate cancer in a patient in need of such treatment comprising administering to the patient a therapeutically effective amount of a compound of Formula (I) and a second drug that is docetaxel.

In another aspect, the present invention provides a method of treating cancer (e.g., melanoma or glioblastoma) in a patient in need of such treatment comprising administering to the patient a therapeutically effective amount of a compound of Formula (I) and temozolomide.

In yet another aspect, the present invention provides a method of treating cancer (e.g., multiple myeloma, MDS) in a patient in need of such treatment comprising administering to the patient a therapeutically effective amount of a compound of Formula (I) and lenalidomide or thalidomide.

In yet another aspect, the present invention provides a method of treating cancer (e.g., non-small cell lung cancer) comprising administering to a patient in need of treatment sequentially first with a therapeutically effective amount of a compound of Formula (I) (e.g., tris(8-quinolinolato)gallium(III)), and then a therapeutically effective amount of erlotinib.

In yet another aspect, the present invention provides a method of treating cancer (e.g., non-small cell lung cancer) comprising administering to a patient in need of treatment sequentially first with a therapeutically effective amount of paclitaxel, and then a therapeutically effective amount of a compound of Formula (I) (e.g., tris(8-quinolinolato)gallium(III)).

Pharmaceutical compositions and kits comprising a therapeutically effective amount of a compound of Formula (I) below, particularly tris(8-quinolinolato)gallium(III), and a second anti-cancer drug as described above are also provided.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a combination index plot illustrating the additive to synergistic activity between tris(8-quinolinolato)gallium(III) and 5-FU in the breast carcinoma cell line ZR-75-1.

FIG. 2 is a combination index plot illustrating the additive to synergistic activity between tris(8-quinolinolato)gallium(III) and paclitaxel in the breast carcinoma cell line ZR-75-1.

FIG. 3 is a combination index plot illustrating the additive to synergistic activity between tris(8-quinolinolato)gallium(III) and 5-FU in the colorectal adenocarcinoma cell line LoVo.

FIG. 4 is a combination index plot illustrating the additive to synergistic activity between tris(8-quinolinolato)gallium(III) and docetaxel in the prostate carcinoma cell line LNCaP-1.

FIG. 5 is a combination index plot illustrating the additive to synergistic activity between tris(8-quinolinolato)gallium(III) and temsirolimus in the lung carcinoma cell line A549.

FIG. 6 is a combination index plot illustrating the additive to synergistic activity between tris(8-quinolinolato)gallium(III) and gemcitabine in the lung carcinoma cell line A549.

FIG. 7 is a combination index plot illustrating the additive to synergistic activity between tris(8-quinolinolato)gallium(III) and temozolomide in the malignant melanoma cell line G361.

FIG. 8 shows the combined growth inhibitory activities of tris(8-quinolinolato)gallium(III) and lenalidomide against the multiple myeloma tumor cell line RPMI-8226. Y axis: % control; X axis: Concentration (μM).

FIG. 9 shows combination index plots illustrating antagonism (circle) between erlotinib and tris(8-quinolinolato)gallium(III) in lung carcinoma cell line A549 treated sequentially first with erlotinib and then with tris(8-quinolinolato)gallium(III); and synergism (square) when treated in the reverse sequence.

FIG. 10 shows combination index plots illustrating antagonism (square) between paclitaxel and tris(8-quinolinolato)gallium(III) in lung carcinoma cell line NCI—H322M treated sequentially with tris(8-quinolinolato)gallium(III) first and then with paclitaxel, but synergism (circle) between the two drugs when used in a reverse sequence.

FIG. 11 shows a combination index plot illustrating the antagonism between doxorubicin and tris(8-quinolinolato)gallium(III) in colon cancer LoVo cell line.

FIG. 12 shows a combination index plot illustrating antagonistic activity between tris(8-quinolinolato)gallium(III) and paclitaxel in the colorectal adenocarcinoma cell line LoVo.

### DETAILED DESCRIPTION OF THE INVENTION

The present invention provides a method of treating cancer by a combination therapy. The method comprises treating a cancer patient in need of treatment with a therapeutically effective amount of (1) a compound of Formula (I)

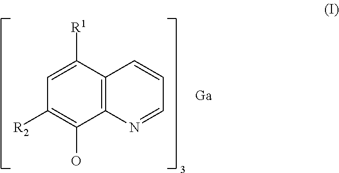

wherein R¹ represents hydrogen, a halogen or a sulfono group SO₃M, in which M is a metal ion, and R² represents hydrogen, or R¹ is Cl and R² is I, or a pharmaceutically acceptable salt thereof; and simultaneously or sequentially (2) a second anti-cancer drug as described below.

As used herein, the phrase “treating . . . with . . . ” means administering a compound to the patient or causing the formation of a compound inside the patient. As used herein, the term “pharmaceutically acceptable salts” refers to the relatively non-toxic, organic or inorganic salts of the active compounds, including inorganic or organic acid addition salts of the compound.

In one aspect, a method of treating breast cancer or colorectal cancer in a patient is provided comprising (1) administering to the cancer patient in need of treatment a therapeutically effective amount of a compound of Formula (I):

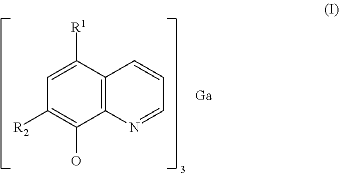

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of 5-fluorouracil or a prodrug thereof (e.g., capecitabine, S1 or tegafur). To put it differently, in accordance with this aspect, the method comprises administering a therapeutically effective amount of a compound of Formula (I) above or a pharmaceutically acceptable salt thereof to a cancer patient who is under treatment of 5-fluorouracil or a prodrug thereof (e.g., capecitabine, S1 or tegafur), or administering a therapeutically effective amount of a 5-fluorouracil or a prodrug thereof (e.g., capecitabine, S1 or tegafur) to a cancer patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in simultaneous or sequential combination with 5-fluorouracil or a prodrug thereof (e.g., capecitabine, S1 or tegafur) for treating breast cancer or colorectal cancer. In another embodiment, the present invention provides a use of 5-fluorouracil or a prodrug thereof (e.g., capecitabine, S1 or tegafur) for the manufacture of a medicament useful in combination with a compound of Formula (I) above or a pharmaceutically acceptable salt thereof for treating breast cancer or colorectal cancer.

In another aspect, a method of treating breast cancer in a patient is provided comprising (1) administering to the cancer patient in need of treatment a therapeutically effective amount of a compound of Formula (I):

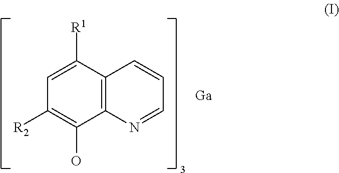

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of paclitaxel or a prodrug thereof (e.g., Abraxane® (paclitaxel protein-bound particles for injectable suspension)). To put it differently, in accordance with this aspect, the method comprises administering a therapeutically effective amount of a compound of Formula (I) above or a pharmaceutically acceptable salt thereof to a breast cancer patient who is under treatment of paclitaxel or a prodrug thereof, or administering a therapeutically effective amount of paclitaxel or a prodrug thereof (e.g., Abraxane® (paclitaxel protein-bound particles for injectable suspension)) to a cancer patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in simultaneous or sequential combination with paclitaxel or a prodrug thereof (e.g., Abraxane® (paclitaxel protein-bound particles for injectable suspension)) for treating breast cancer. In another embodiment, the present invention provides a use of paclitaxel or a prodrug thereof for the manufacture of a medicament useful in combination (simultaneous or sequential) with a compound of Formula (I) above or a pharmaceutically acceptable salt thereof for treating breast cancer.

In another aspect, a method of treating prostate cancer in a patient is provided comprising (1) administering to the cancer patient in need of treatment a therapeutically effective amount of a compound of Formula (I):

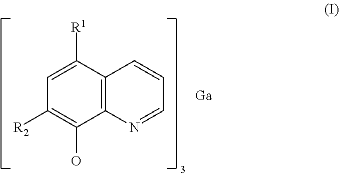

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of docetaxel. To put it differently, in accordance with this aspect, the method comprises administering a therapeutically effective amount of a compound of Formula (I) above or a pharmaceutically acceptable salt thereof to a prostate cancer patient who is under treatment of docetaxel or a prodrug thereof, or administering a therapeutically effective amount of docetaxel to a cancer patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in combination (simultaneous or sequential) with docetaxel for treating prostate cancer. In another embodiment, the present invention provides a use of docetaxel for the manufacture of a medicament useful in combination (simultaneously or sequentially) with a compound of Formula (I) above or a pharmaceutically acceptable salt thereof for treating prostate cancer.

In another aspect, a method of treating lung cancer (e.g., non-small cell lung cancer or small cell lung cancer) in a patient is provided comprising (1) administering to the cancer patient in need of treatment a therapeutically effective amount of a compound of Formula (I):

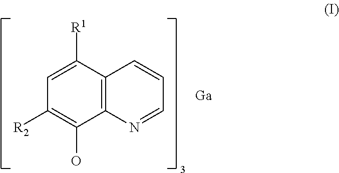

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of gemcitabine. To put it differently, in accordance with this aspect, the method comprises administering a therapeutically effective amount of a compound of Formula (I) above or a pharmaceutically acceptable salt thereof to a lung cancer patient who is under treatment of gemcitabine, or administering a therapeutically effective amount of gemcitabine to a lung cancer patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in combination (simultaneously or sequentially) with gemcitabine for treating lung cancer. In another embodiment, the present invention provides use of gemcitabine for the manufacture of a medicament useful in combination (simultaneously or sequentially) with a compound of Formula (I) above or a pharmaceutically acceptable salt thereof for treating lung cancer.

In another aspect, a method of treating cancer (e.g., non-small cell lung cancer, renal cell carcinoma, or neuroendocrine tumors (e.g., pancreatic neuroendocrine tumors)) in a patient is provided comprising (1) administering to the cancer patient in need of treatment a therapeutically effective amount of a compound of Formula (I):

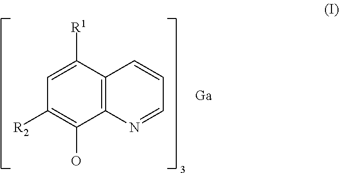

wherein R¹ represents hydrogen, a halogen or a sulfono group SO₃M, in which M is a metal ion, and R² represents hydrogen, or R¹ is Cl and R² is I, or a pharmaceutically acceptable salt thereof; and simultaneously or sequentially (2) administering to said cancer patient a therapeutically effective amount of temsirolimus. To put it differently, in accordance with this aspect, the method comprises administering a therapeutically effective amount of a compound of Formula (I) above or a pharmaceutically acceptable salt thereof to a cancer patient (e.g., non-small cell lung cancer, renal cell carcinoma, or neuroendocrine tumors (e.g., pancreatic neuroendocrine tumors)) who is under treatment of temsirolimus, or administering a therapeutically effective amount of temsirolimus to a cancer patient (e.g., non-small cell lung cancer, renal cell carcinoma, or neuroendocrine tumors (e.g., pancreatic neuroendocrine tumors)) who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in combination (simultaneously or sequentially) with temsirolimus for treating cancer (e.g., non-small cell lung cancer, renal cell carcinoma, or neuroendocrine tumors (e.g., pancreatic neuroendocrine tumors)). In another embodiment, the present invention provides use of temsirolimus for the manufacture of a medicament useful in combination (simultaneously or sequentially) with a compound of Formula (I) above or a pharmaceutically acceptable salt thereof for treating cancer (e.g., non-small cell lung cancer, renal cell carcinoma, or neuroendocrine tumors (e.g., pancreatic neuroendocrine tumors)).

In another aspect, a method of treating cancer (e.g., melanoma or brain tumor such as glioblastoma) in a patient is provided comprising (1) administering to the cancer patient in need of treatment a therapeutically effective amount of a compound of Formula (I):

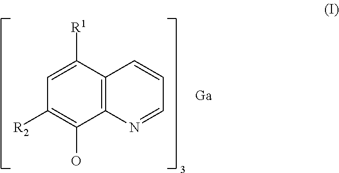

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of temozolomide. To put it differently, in accordance with this aspect, the method comprises administering a therapeutically effective amount of a compound of Formula (I) above or a pharmaceutically acceptable salt thereof to a cancer patient (diagnostic with e.g., melanoma or brain tumor such as glioblastoma) who is under treatment of temozolomide, or administering a therapeutically effective amount of temozolomide to a melanoma patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in combination (simultaneously or sequentially) with temozolomide for treating cancer (e.g., melanoma or brain tumor such as glioblastoma). In another embodiment, the present invention provides use of temozolomide for the manufacture of a medicament useful in combination (simultaneously or sequentially) with a compound of Formula (I) above or a pharmaceutically acceptable salt thereof for treating cancer (e.g., melanoma or brain tumor such as glioblastoma).

In another aspect, a method of treating cancer (e.g., multiple myeloma or myelodysplastic syndromes (MDS)) in a patient is provided comprising (1) administering to the cancer patient in need of treatment a therapeutically effective amount of a compound of Formula (I):

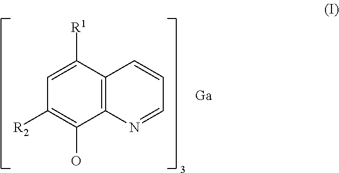

wherein R¹ represents hydrogen, a halogen or a sulfono group SO₃M, in which M is a metal ion, and R² represents hydrogen, or R¹ is Cl and R² is I, or a pharmaceutically acceptable salt thereof and simultaneously or sequentially (2) administering to said cancer patient a therapeutically effective amount of lenalidomide or thalidomide. To put it differently, in accordance with this aspect, the method comprises administering a therapeutically effective amount of a compound of Formula (I) above or a pharmaceutically acceptable salt thereof to a cancer patient (e.g., having multiple myeloma or MDS) who is under treatment of lenalidomide or thalidomide, or administering a therapeutically effective amount of lenalidomide or thalidomide to a cancer (e.g., multiple myeloma or MDS) patient who is under treatment of a compound of Formula (I) above or a pharmaceutically acceptable salt thereof. Also put it differently, the present invention provides a use of a compound of Formula (I) above or a pharmaceutically acceptable salt thereof for the manufacture of a medicament useful in combination (simultaneously or sequentially) with lenalidomide or thalidomide for treating cancer (e.g., multiple myeloma or MDS). In another embodiment, the present invention provides use of lenalidomide or thalidomide for the manufacture of a medicament useful in combination (simultaneously or sequentially) with a compound of Formula (I) above or a pharmaceutically acceptable salt thereof for treating cancer (e.g., multiple myeloma or MDS).

In yet another aspect, the present invention provides a method of treating cancer (e.g, non-small cell lung cancer) comprising administering to the cancer patient in need of treatment sequentially, first a therapeutically effective amount of a compound of Formula (I):

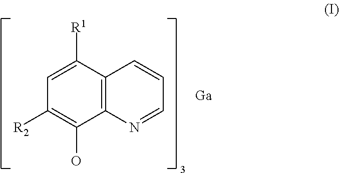

wherein R¹ represents hydrogen, a halogen or a sulfono group SO₃M, in which M is a metal ion, and R² represents hydrogen, or R¹ is Cl and R² is I, or a pharmaceutically acceptable salt thereof (e.g., tris(8-quinolinolato)gallium(III)), and then a therapeutically effective amount of erlotinib. To put it differently, the present invention provides a use of a compound of Formula (I) above or a pharmaceutically acceptable salt thereof (e.g., tris(8-quinolinolato)gallium(III)) for the manufacture of a medicament useful in a sequential combination with erlotinib for treating cancer (e.g., non-small cell lung cancer), wherein the compound of Formula (I) above or a pharmaceutically acceptable salt thereof (e.g., tris(8-quinolinolato)gallium(III)) is administered first and erlotinib is administered second.

In yet another aspect, the present invention provides a method of treating cancer (e.g., non-small cell lung cancer) comprising administering to the cancer patient in need of treatment sequentially, first a therapeutically effective amount of paclitaxel or a prodrug thereof (e.g., Abraxane® (paclitaxel protein-bound particles for injectable suspension)), and then a therapeutically effective amount of a compound according to Formula (I):

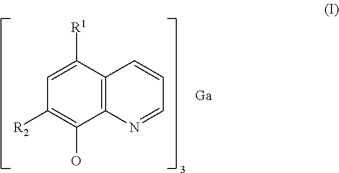

wherein R¹ represents hydrogen, a halogen or a sulfono group SO₃M, in which M is a metal ion, and R² represents hydrogen, or R¹ is Cl and R² is I, or a pharmaceutically acceptable salt thereof (e.g., tris(8-quinolinolato)gallium(III)). To put it differently, the present invention provides a use of a compound of Formula (I) above or a pharmaceutically acceptable salt thereof (e.g., tris(8-quinolinolato)gallium(III)) for the manufacture of a medicament useful in sequential combination with paclitaxel or a prodrug thereof (e.g., Abraxane® (paclitaxel protein-bound particles for injectable suspension)) for treating cancer (e.g., non-small cell lung cancer), wherein paclitaxel or a prodrug thereof (e.g., Abraxane® (paclitaxel protein-bound particles for injectable suspension)) is administered first and the compound of Formula (I) above or a pharmaceutically acceptable salt thereof (e.g., tris(8-quinolinolato)gallium(III)) is administered second.

In preferred embodiments of the different aspects described above, the compound of Formula (I) is tris(8-quinolinolato)gallium(III). Tris(8-quinolinolato)gallium(III), also known as tris-(8-hydroxyquinoline)gallium, is a gallium complex compound first made by Professor Bernhard Keppler and is disclosed in, e.g., U.S. Pat. No. 5,525,598.

Thus, in these various embodiments in accordance with the present invention, a patient having cancer (e.g., the specific types of cancer described above) is identified or diagnosed, and such patient is treated with a therapeutically effective amount of an anti-cancer drug such as 5-FU or a prodrug thereof (e.g., capecitabine, tegafur, S1), paclitaxel or a prodrug thereof (e.g., Abraxane® (paclitaxel protein-bound particles for injectable suspension)), docetaxel, gemcitabine, temsirolimus, temozolomide, lenalidomide, thalidomide and erlotinib in combination with (simultaneously or sequentially) a therapeutically effective amount of a compound of Formula (I) such as tris(8-quinolinolato)gallium(III).

In the combination therapy methods of the present invention, the compound of Formula (I) such as tris(8-quinolinolato)gallium(III) and the other anti-cancer drug(s) can be administered at about the same time, or separately according to their respective dosing schedules or regimens. When administered at about the same time, the gallium compound and the other anti-cancer drug(s) can be administered in the same pharmaceutical composition or in separate dosage unit forms. For example, in one embodiment of the combination method of the present invention, the compound tris(8-quinolinolato)gallium(III) can be administered, e.g., orally at a dosing of from 0.1 mg to 3000 mg at, e.g., four times a day, while the other anti-cancer drugs can be administered at a dose and dosing schedule as provided in the FDA-approved prescribing information. In the sequential combination therapies discussed above, preferably the drugs in sequential combination are administered according to their pharmacokinetic profiles such that the second drug is administered after the plasma level of the first drug is substantially reduced or removed. The pharmacokinetic parameters of tris(8-quinolinolato)gallium(III) is disclosed in Hofheinz et al. International Journal of Clinical Pharmacology and Therapeutics, 43 (12): 590-591 (2005). The pharmacokinetic profiles of the other drugs discussed above are generally known in the art.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, adverse events, and the like, as will be apparent to a skilled artisan. The amount of administration can be adjusted as the various factors change over time.

The pharmaceutical compounds in the method of present invention can be administered in any suitable unit dosage forms. Suitable oral formulations can be in the form of tablets, capsules, suspension, syrup, chewing gum, wafer, elixir, and the like. Pharmaceutically acceptable carriers such as binders, excipients, lubricants, and sweetening or flavoring agents can be included in the oral pharmaceutical compositions. If desired, conventional agents for modifying tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

For injectable formulations, the pharmaceutical compositions can be in lyophilized powder in admixture with suitable excipients in a suitable vial or tube. Before use in the clinic, the drugs may be reconstituted by dissolving the lyophilized powder in a suitable solvent system to form a composition suitable for intravenous or intramuscular injection.

In accordance with another aspect of the present invention, a pharmaceutical composition is provided, comprising a therapeutically effective amount of a compound of Formula (I) such as tris(8-quinolinolato)gallium(III) as well as a therapeutically effective amount of a second drug chosen from the group of capecitabine, tegafur, S1, paclitaxel, a prodrug of paclitaxel (e.g., Abraxane® (paclitaxel protein-bound particles for injectable suspension), docetaxel, gemcitabine, temsirolimus, temozolomide, lenalidomide and thalidomide. In preferred embodiments, the pharmaceutical composition comprises a therapeutically effective amount of a compound of Formula (I) such as tris(8-quinolinolato)gallium(III) and a therapeutically effective amount of capecitabine, tegafur, S1, temsirolimus, temozolomide, lenalidomide or thalidomide. In one embodiment, the composition can be in an orally administrable form (e.g., tablet or capsule or syrup etc.) with a therapeutically effective amount (e.g., 0.1 mg to about 3000 mg) of tris(8-quinolinolato)gallium(III) and a therapeutically effective amount (e.g., from 0.1 mg to about 2000 mg) of a second anti-cancer drug as described above. In a specific embodiment, the second anti-cancer drug is temsirolimus, temozolomide, lenalidomide or thalidomide, and the composition includes temozolomide at 5 mg to about 250 mg, or 25 mg of temsirolimus, or 50 mg to 250 mg of lenalidomide, or 5 mg to 25 mg of thalidomide, or 10 mg to 500 mg of capecitabine, or 10-200 mg of S1, or 10-200 mg of tegafur.

In accordance with another aspect of the present invention, a pharmaceutical kit is provided comprising, in a compartmentalized container, (1) a unit dosage form of a compound of Formula (I) such as tris(8-quinolinolato)gallium(III); and (2) a unit dosage form of a second drug chosen from the group of capecitabine, tegafur, S1, paclitaxel, a prodrug of paclitaxel (e.g., Abraxane® (paclitaxel protein-bound particles for injectable suspension), docetaxel, gemcitabine, temsirolimus, temozolomide, lenalidomide and thalidomide, preferably from the group of tegafur, S1, temsirolimus, temozolomide, lenalidomide and thalidomide. As will be apparent to a skilled artisan, the amount of a therapeutic compound in the unit dosage form is determined by the dosage to be used on a patient in the method of the present invention. In one embodiment of the kit, tris(8-quinolinolato)gallium(III) is in a tablet or capsule or any other suitable form at an amount of, e.g., 0.1 mg to about 3000 mg per unit dosage form. The kit further includes a second anti-cancer drug as described above in a unit dosage of from about 0.1 mg to about 2000 mg in a tablet or capsule form or in lyophilized powder. In a specific embodiment, the second anti-cancer drug is temsirolimus, temozolomide, lenalidomide or thalidomide, and the kit includes a capsule of temozolomide of 5 mg to about 250 mg, or a vial of 25 mg of temsirolimus in lyophilized powder, or a capsule of 50 mg to 250 mg of lenalidomide, or a capsule of 5 mg to 25 mg of thalidomide, or 10 mg to 500 mg of capecitabine in an oral dosage form such as tablet or capsule, or 10-200 mg of S1 in an oral dosage form, or 10-200 mg of tegafur in an oral dosage form. Optionally, the kit further comprises instructions for using the kit in the combination therapy method in accordance with the present invention.

### Example 1

Cultures of human tumor cell lines A549, LNCap clone FGC, LoVo, ZR-75-1, and G361 were established using standard in vitro culture methods and supplier recommended media and supplements in 175 cm² Greiner® or Corning® tissue culture-treated flasks. All cell cultures were incubated in a humidified 37° C., 5% CO₂, 95% air environment. The cells were sub-cultured regularly to maintain log phase growth. On the day of EC₅₀ plate seeding, the cells for each line were processed and seeded into 96-well cell culture-treated plates one cell line at a time. The cells were removed from their culture flasks using trypsin solution pooled in a sterile conical tube and centrifuged at 350×g for 5 minutes at room temperature. Pelleted cells were re-suspended in complete media and then counted with a Neubauer Bright-Line® hemacytometer and trypan blue viability stain. The cell suspensions were diluted (based on live cell counts) using complete media to yield a final suspension density (cells/ml) based on previously determined seeding densities for each cell line for a 72 hour 96-well plate assay. The tissue culture treated plates for EC₅₀ testing were seeded at a density specified below in Table 1 and incubated overnight at 37° C. in a 5% CO₂, 95% air humidified atmosphere to allow the cells to attach.

TABLE 1
Seeding Density for EC₅₀ Assay

| | | Cells/well |
|---|---|---|
| Cell Line | Type | (×10³) |
| A549 | lung cancer | 2.5 |
| LNCaP | prostate cancer | 4.0 |
| LoVo | colorectal cancer | 12.0 |
| G361 | malignant melanoma cancer | 2.5 |
| ZR-75-1 | breast cancer | 3.0 |

For each single agent or combination of test agents, the top concentration mixture (2× final treatment concentration) was made in sterile 1.5 ml microcentrifuge tubes and then directly transferred to the first well of the treatment dilution plates.

Tris(8-quinolinolato)gallium(III) (a fine, medium yellow colored powder; lot Q04014, M.W. 502.19, >95% parent) was obtained from Niiki Pharma, Inc. in Parafilm®-sealed screw capped clear glass vials and stored at −20° C. in a covered box to prevent exposure to light. 5-Fluororuracil was manufactured by TEVA Parenteral Medicines and supplied in vials at a concentration of 50 mg/ml (384.4 mM) in aqueous solution. Docetaxel manufactured by Fluka was weighed out (1.6 mg) and a 2,000 μM solution was made by adding 0.990 ml 100% DMSO and intermittently vortexing for 1-15 seconds. This was further diluted in 100% DMSO to make a 40 μM stock solution (10 μl of 2,000 μM docetaxel+490 μA DMSO). 5.8 mg of Gemcitabine (manufactured by Eli Lilly and Company) was weighed out and a 50 mM clear, and colorless stock solution was made by adding 188 μl of sterile water. This was further diluted 1,000× in complete media to yield a 50 μM stock solution (10 μl of 50 mM gemcitabine+9.990 ml media).

Paclitaxel (a fine, white powder; lot TECH600600-A, M.W. 853.9, 99% parent) was manufactured by Cedarburg Hauser and supplied in an amber glass screw cap vial sealed with Parafilm®. It was stored at −20° C. in a covered box to protect from exposure to light. Paclitaxel (2 mM clear, colorless stock solution in 100% DMSO) was made previously and stored at −20° C. in a covered box to protect from exposure to light. One vial of 2 mM paclitaxel stock solution was quickly thawed in hand just prior to use. The 2 mM stock solution was diluted 1000× in complete media (10 μL 2 mM Paclitaxel+9.990 mL media) to yield a 2 μM working stock solution.

Temsirolimus (a white, medium particulate powder; lot BTM-104, M.W. 1030.29, >99% purity) was obtained from LC Laboratories (product # T-8040) and supplied in an amber glass vial. It was stored in the dark at −20° C. and sealed with Parafilm® to limit exposure to light and humidity. Temsirolimus (6.1 and 8.1 mg) was weighed out and 100 and 200 mM clear, colorless stock solutions were made by adding 59.2 and 39 μL, respectively of 100% DMSO.

Temozolomide (a very light pink tinted granular powder; lot 7BTR042, M.W. 194.15, 55.84% parent) was manufactured by the Schering Corporation and supplied in an amber glass vial. It was stored in the dark at room temperature and sealed with Parafilm® to limit exposure to light and humidity. Temozolomide (20.7 mg) was weighed out and a 400 mM white, cloudy suspension was made by adding 149 μL of 100% DMSO and brief sonication (˜10-20 seconds) in a sonicating water bath without heat.

The antiproliferative activity of the test agents was evaluated using the MTT Cell Proliferation Assay Kit (ATCC catalog #30-1010K). The MTT assay is based on the reduction of yellow tetrazolium MTT (3-(4,5-dimethylthiazolyl-2)-2,5-diphenyltetrazolium bromide) by metabolically active cells forming purple formazan crystals. The purple formazan is solublized with detergent and quantified spectrophotometrically at 570 nm. Cells in the log phase of growth were seeded at the indicated densities listed in Table 1 above into 96-well culture treated plates in 0.1 mL of complete media in all wells except for one column reserved for the media only control. The cells were allowed to attach during an overnight incubation prior to treating with test agents. Test agents were serially diluted in complete culture media (+1% DMSO where appropriate) and added to each well in a volume of 0.1 mL for a total final volume of 0.2 mL/well (0.5% DMSO final, where used). Cells were exposed to test agents for 72 hours. Following the exposure to test agents, 0.1 mL of culture supernatant was carefully removed from all wells of each plate and 0.01 mL of MTT reagent was added to each well. The plates were returned to the incubator for four hours. Following the incubation period, kit supplied detergent reagent (0.1 mL) was added to all wells. The plates were wrapped in plastic wrap to prevent evaporation and allowed to sit at room temperature in the dark overnight. The absorbance at 570 nm was measured the following day using a SpectraMAX Plus plate reader (Molecular Devices). Absorbance values were converted to Percent of Control and plotted against test agent concentrations for EC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration vs. Percent of Control were analyzed using the 4-parameter equation to obtain EC₅₀ values and other parameters that describe the sigmoidal dose response curve.

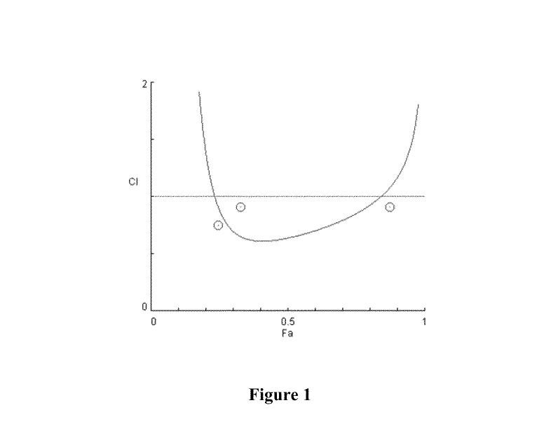

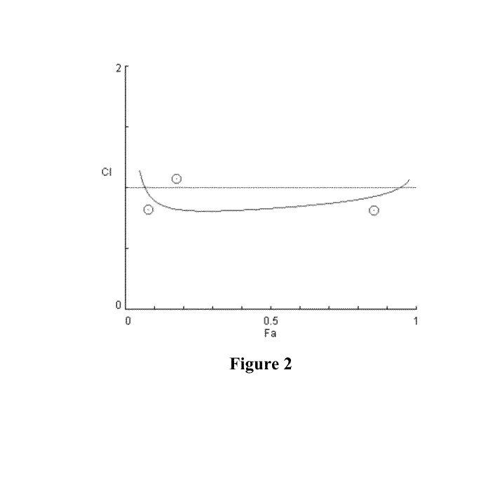

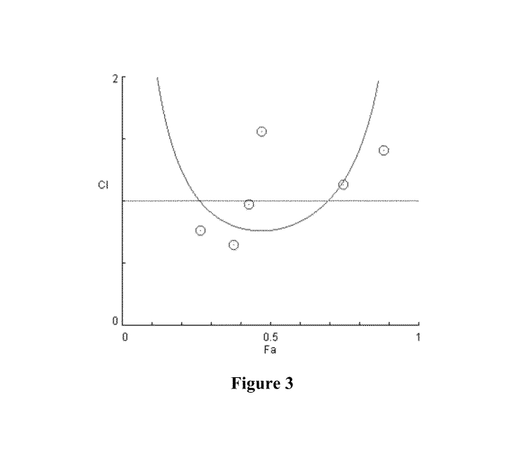

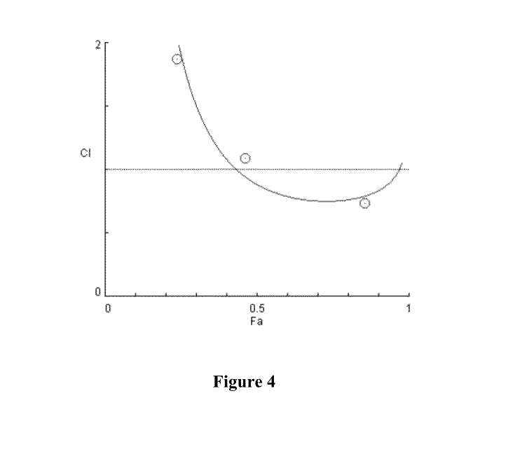

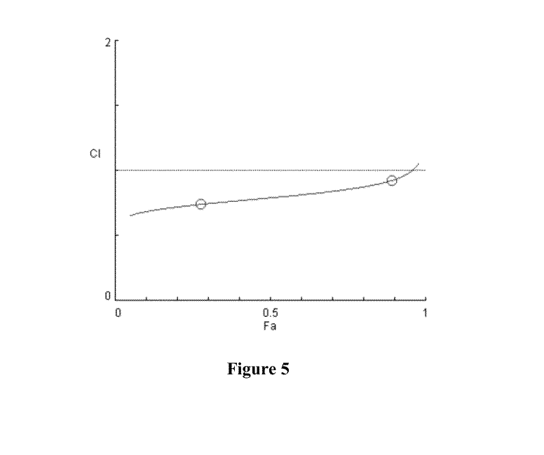

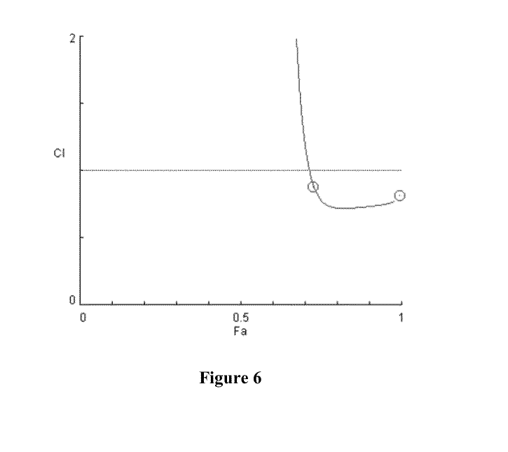

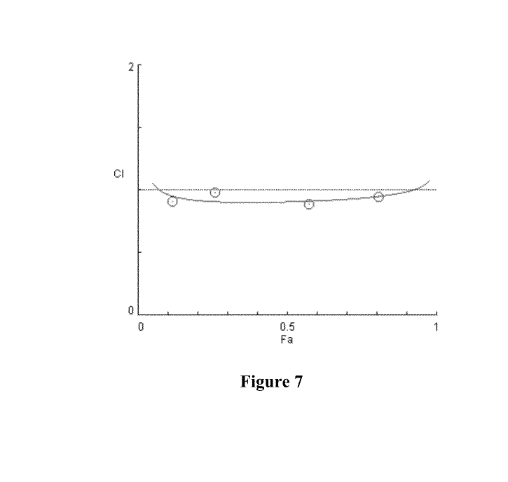

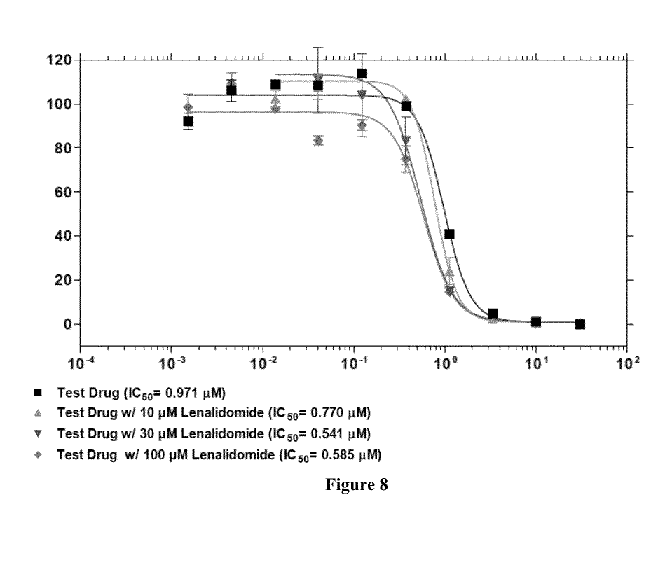

Combination data was analyzed using CompuSyn® software to calculate Combination Index (CI) values to assess synergy. The Fractional Affect (Fa) was calculated from the Pecent of Control (from SoftMax® Pro) using the formula: 1−(Percent Control/100). The dosage, fractional affect and molar ratio of compounds tested in combination were entered into the CompuSyn® software for evaluation of the presence/absence of synergy. CompuSyn assigns a Combination Index (CI) value which rates the level of compounds' affect on proliferation. CI values below 1 indicate the presence of synergy and CI values above 1 indicate antagonism. CI values close to 1 indicate an additive affect. See Chou, Pharmacol. Rev., 58(3):621-81 (2006). Table 2 below summarizes the CI values of the synergistic combinations. FIGS. 1-8 are combination index plots illustrating the synergistic activity between tris(8-quinolinolato)gallium(III) and the above other drugs.

TABLE 2
Combination Index Values

| | | Combination Index |
|---|---|---|
| Combination | Cell Line | (CI) Values* |
| Test drug + docetaxel | LNCaP-1 | 0.752 |
| Test drug + 5-fluorouracil | Lovo | 0.766 |
| | ZR-75-1 | 0.638 |
| Test drug + gemcitabine | A549 | 0.778 |
| Test drug + paclitaxel | ZR-75-1 | 0.831 |
| Test drug + temozolomide | G361 | 0.906 |
| Test drug + temsirolimus | A549 | 0.791 |

*0.1-0.90 = Synergism;
0.90-1.10 = Additive;
1.10-10 = Antagonism.

### Example 2

The antiproliferative activity of tris(8-quinolinolato)gallium(III) and lenalidomide against the human multiple myeloma tumor cell line RPMI 8226 was determined with Promega's Cell Titer-Glo® assay. The human tumor cells were placed in a 96-well microculture plate at the appropriate density for 96 hours of total growth time. After 24 hours of incubation in a humidified incubator at 37° C. with 5% CO₂ and 95% air, serially diluted test agents in growth medium were added to each well. After 96 total hours of culture in a CO₂ incubator, the plates were processed with Cell Titer-Glo (Promega #G7571) according to manufacturer's instructions. Luminescence was detected using a Tecan GENios microplate reader. Percent inhibition of cell growth was calculated relative to untreated control wells. All tests were performed in duplicate at each concentration level. The IC₅₀ value for the test agents was estimated using Prism 3.03 by curve-fitting the data using the following four parameter-logistic equation:

$$Y = \frac{Top - Bottom}{1 + {( \frac{X}{{IC}_{50}} )}^{n}} + Bottom$$

where Top is the maximal % of control absorbance, Bottom is the minimal % of control absorbance at the highest agent concentration, Y is the % of control absorbance, X is the agent concentration, IC50 is the concentration of agent that inhibits cell growth by 50% compared to the control cells, and n is the slope of the curve.

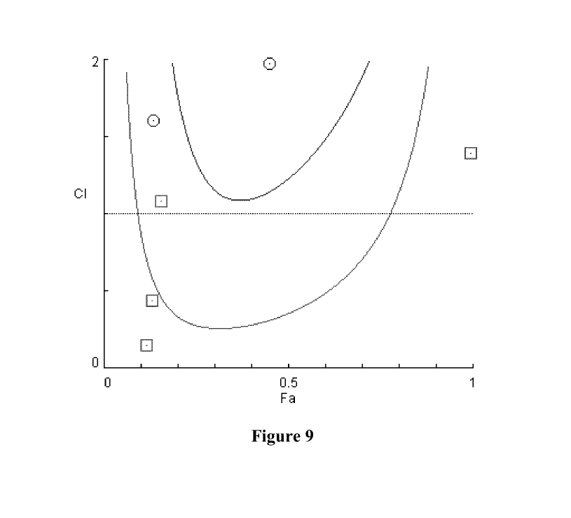

The same Promega Cell Titer-Glo® assay described above was used in the combination study. The IC₅₀ values of tris(8-quinolinolato)gallium(III) and lenalidomide were used to determine appropriate drug ratios and concentration ranges for a combination study based on the constant ratio design of Chou-Talalay. Drug ratios were equivalent to the ratio of respective IC₅₀ of agents being combined. Assuming a combination response of near additivity, drug concentrations bracketed the sum of one half of the respective IC₅₀'s with serial dilutions selected based upon the inhibition curves of the agents being combined, (typically 1.5 fold dilutions) with a total of seven drug concentrations. After 96 hours, the cell number was determined with the Cell Titer Glo® assay as described above. FIG. 9 shows the combined growth inhibitory activities of tris(8-quinolinolato)gallium(III) and lenalidomide against the multiple myeloma tumor cell line RPMI-8226. Y axis: % control X axis: Concentration (μM). As shown in FIG. 9, adding lenalinomide at a constant amount potentiates the tris(8-quinolinolato)gallium(III) effect. The IC₅₀ goes from ˜0.9 with tris(8-quinolinolato)gallium(III) alone down to 0.5 with lenalidomide.

### Example 3

This data illustrates that the sequence in which tris(8-quinolinolato)gallium(III) is administered in combination with erlotinib or paclitaxel is important in determining whether it will be a synergistic or antagonistic combination. Note: Simultaneous incubation of tris(8-quinolinolato)gallium(III) with either of these drugs gave an antagonistic effect (see Tables 4 and 5 below; graph not shown).

A549 and NCI-H322M human tumor cell lines were obtained from the American Type Culture Collection (ATCC) and the National Cancer Institute (NCI), respectively. Cell cultures were established using standard in vitro culture methods and cell line supplier's recommended media and supplements in 175 cm² Greiner® tissue culture-treated flasks. All cell cultures were incubated in a humidified 37° C., 5% CO2, 95% air environment. The cells were sub-cultured regularly to maintain log phase growth. On the day of EC50 plate seeding, the cells for each line were processed and seeded into 96-well cell culture-treated plates one cell line at a time.

TABLE 3
Seeding Density for EC₅₀ Assay

| Cell Line | Cells/Well (×10³) |
|---|---|
| A549 | 3 |
| NCI-H322M | 10 |

For initial treatments with tris(8-quinolinolato)gallium(III), 42 mg was weighed out and a 200 mM suspension was made using 100% DMSO. 100 μl of the suspension was transferred directly to 9.9 ml of complete media for each cell line for a 1:100 dilution. This was then serially diluted 1:4 in complete media containing 0.1% DMSO across nine wells of a 96-well dilution plate for a total of ten concentrations ranging from 2,000-0.008 μM tris(8-quinolinolato)gallium(III)/0.1% DMSO. For the 48 hour treatment with tris(8-quinolinolato)gallium(III), 39 mg was weighed out and a uniform suspension was made using 100% DMSO. 100 μl of this suspension was transferred directly to 9.9 ml of complete media for both cell lines for a 1:100 dilution. This was then serially diluted 1:4 in complete media containing 0.1% DMSO, across nine wells of a 96-well dilution plate for a total of ten concentrations ranging from 2,000-0.00804 tris(8-quinolinolato)gallium(III).

Erlotinib was obtained from LC Laboratories. A 40 mM stock solution was made. This solution was immediately diluted 1:80 into cell line-specific complete media (100 μl 40 mM erlotinib into 7.9 ml media). This was then serially diluted 1:4 in complete media containing 1.25% DMSO, across nine wells of a 96-well dilution plate for a total of ten concentrations ranging from 250-0.002 μM.

Paclitaxel was manufactured by Cedarburg Hauser. A 2,000 μM clear, colorless stock solution was made using 100% DMSO. This was diluted 1,000× in complete media for a top concentration of 2,000 nM (0.1% DMSO) in the first well of a 96-well dilution plate. This was then serially diluted 1:4 in complete media across nine wells of the dilution plate for a total of ten concentrations ranging from 2,000-0.008 nM.

The antiproliferative activity of the test agents was evaluated using the MTT Cell Proliferation Assay Kit (ATCC catalog #30-1010K). Cells in the log phase of growth were seeded at the indicated densities into 96-well culture treated plates in 0.1 ml of complete media in all wells except column 12, which was reserved for the media only control (blank). The cells were allowed to attach during an overnight incubation prior to treating with test agents. Test agents were serially diluted in complete culture media (with the addition of DMSO where appropriate) and added to each well in a volume of 0.1 ml for a total final volume of 0.2 ml/well. Cells were exposed to test agents for a total of four days (96 hours). Following the exposure to test agents, 0.1 ml of culture supernatant was carefully removed from all wells of each plate and 0.01 ml of MTT reagent was added to each well. The plates were returned to the incubator for four hours. Following the incubation period, kit supplied detergent reagent (0.1 ml) was added to all wells. The plates were wrapped in plastic wrap to prevent evaporation and allowed to sit at room temperature in the dark overnight. The absorbance at 570 nm was measured the following day using a SpectraMAX Plus plate reader (Molecular Devices).

Absorbance values were converted to Percent of Control and plotted against test agent concentrations for EC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration vs. Percent of Control were analyzed using the 4-parameter equation to obtain EC₅₀ values and other parameters that describe the sigmoidal dose response curve.

Combination data were analyzed using CompuSyn® software to calculate Combination Index (CI) values to assess the synergy of test agents. The Fractional Affect (Fa) was calculated from the Percent of Control (from SoftMax® Pro) using the formula: 1−(Percent Control/100). The dosage, fractional affect and molar ratio of compounds tested in combination were entered into the CompuSyn® software for evaluation of the presence/absence of synergy. Data points for use in CompuSyn® analysis were selected from the dose response curve transition area (between the effect and no effect regions) of the treatment response graph of the EC₅₀ SoftMax Pro® data. CompuSyn® assigns a Combination Index (CI) value which rates the level of a combination of compounds' effect on proliferation. CI values below 1 indicate the presence of synergy and CI values above 1 indicate antagonism. CI values close to 1 indicate an additive affect.

TABLE 4
Combination effect of
tris(8-quinolinolato)gallium(III) (“test drug”) and erlotinib

| Combination | Cell line | Effect | CI |
|---|---|---|---|
| Test Drug 48 hrs, | A549 (lung) | Synergism | 0.356 |
| wash, | | | |
| Erlotinib 48 hrs | | | |
| Erlotinib 48 hrs, | A549 (lung) | Antagonism | 1.23 |
| wash, | | | |
| Test Drug 48 hrs | | | |
| Simultaneous Incubation of | A549 (lung) | Antagonism | 1.26 |
| Erlotinib and Test Drug for | | | |
| 72 hours | | | |

FIG. 9 shows a combination index plots illustrating the antagonism (circle) between erlotinib and tris(8-quinolinolato)gallium(III) in lung carcinoma cell line A549 treated with erlotinib first for 48 hours, washed, and then treated with tris(8-quinolinolato)gallium(III) for 48 hours, and the synergism (square) between the two drugs in A549 cell line treated with tris(8-quinolinolato)gallium(III) first for 48 hours, washed, and then treated with erlotinib for 48 hours. This demonstrates the importance of correct treatment sequence.

TABLE 5
Combination effect of
tris(8-quinolinolato)gallium(III) (“test drug”) and paclitaxel

| Combination | Cell line | Effect | CI |
|---|---|---|---|
| Paclitaxel 24 hrs, Wash, | NCI-H322M | Synergism | 0.353 |
| Test Drug 48 hrs, | (lung) | | |
| Wash, | | | |
| Fresh media 24 hrs | | | |
| Test Drug 48 hrs, | NCI-H322M | Antagonism | 2.05 |
| Wash, | (lung) | | |
| Paclitaxel 24 hrs, | | | |
| Wash, | | | |
| Fresh media 24 hrs | | | |
| Simultaneous Incubation of | NCI-H322M | Antagonism | 1.59 |
| Test Drug and Paclitaxel for | (lung) | | |
| 72 hours | | | |

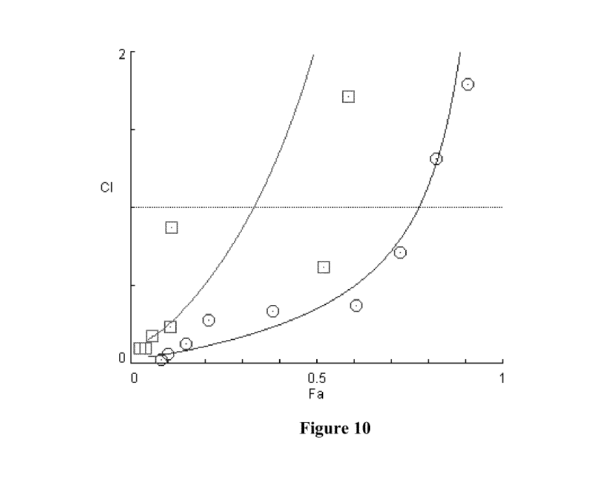

FIG. 10 shows a combination index plots illustrating the antagonism (square) between paclitaxel and tris(8-quinolinolato)gallium(III) in lung carcinoma cell line NCI-H322M treated with tris(8-quinolinolato)gallium(III) first for 48 hours, washed, then treated with paclitaxel for 24 hours, washed, and then cultured in fresh media for 24 hours, and the synergism (circle) between the two drugs in NCI-H322M cell line treated with paclitaxel first for 24 hours, washed, and then treated with tris(8-quinolinolato)gallium(III) for 48 hours, and washed and then cultured in fresh media for 24 hours.

### Comparative Examples

Human tumor cell line LoVo was obtained from the American Type Culture Collection (ATCC), and cultured in medium and under conditions as recommended by the supplier. For a 72 hour 96-well plate assay, the cells were seeded at 12.0×10³ per well and incubated overnight at 37° C. in a 5% CO₂, 95% air humidified atmosphere to allow the cells to attach. Test agents such as tris(8-quinolinolato)gallium(III) and paclitaxel were prepared as described above in Example 3. Doxorubicin was obtained from Teva Parenteral Medicines and a 3.45 mM aqueous stock solution was directly diluted in complete media.

The antiproliferative EC₅₀ activity of the test agents was evaluated using the MTT Cell Proliferation Assay Kit (ATCC catalog #30-1010K), and the data was analyzed as described above in other examples. Table 6 below summarizes the CI values of the combinations.

TABLE 6
Combination Index Values

| | | Combination Index |
|---|---|---|
| Combination | Cell Line | (CI) Values* |
| Test drug + doxorubicin | LoVo | 1.19 |
| Test drug + paclitaxel | LoVo | 5.98 |

*0.1-0.90 = Synergism;
0.90-1.10 = Additive;
1.10-10 = Antagonism.

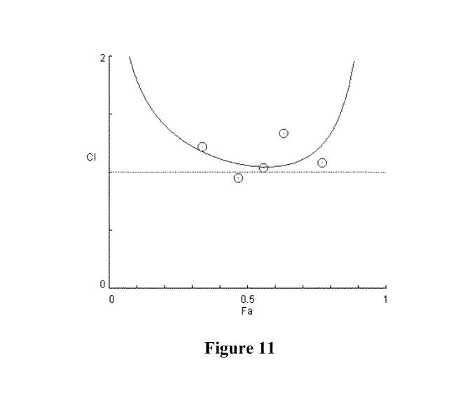

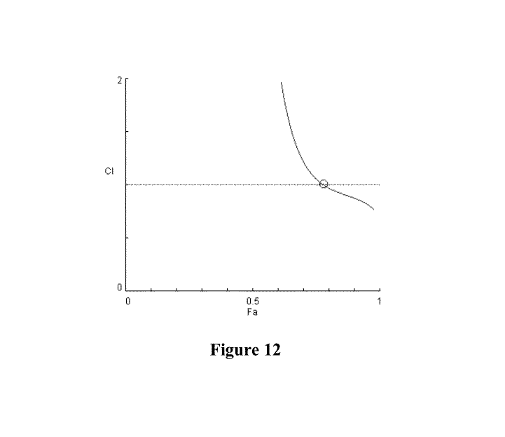

FIG. 11 shows combination index plot illustrating the antagonism between doxorubicin and tris(8-quinolinolato)gallium(III) in colon cancer LoVo cell line. FIG. 12 shows combination index plot illustrating antagonistic activity between tris(8-quinolinolato)gallium(III) and paclitaxel in the colorectal adenocarcinoma cell line LoVo.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

1-18. (canceled)

19. A method for treating or preventing cancer, said method comprising administering to a patient in need thereof a therapeutically effective amount of tris(8-quinolinolato)gallium(III), or a pharmaceutically acceptable salt thereof, in combination with one or more agents chosen from the group of 5-fluorouracil or a prodrug thereof, paclitaxel, Abraxane, docetaxel, gemcitabine, temsirolimus, temozolomide, erlotinib, lenalidomide and thalidomide.

20. The method of claim 19, wherein the 5-fluorouracil or prodrug thereof is selected from the group consisting of capecitabine, S1, and tegafur.

21. A method for treating cancer, comprising administering to a patient in need of treatment of such cancer, simultaneously or sequentially, a therapeutically effective amount of (1) tris(8-quinolinolato)gallium(III), or a pharmaceutically acceptable salt thereof, and (2) temsirolimus.

22. The method of claim 21, wherein said cancer is lung cancer or renal cancer.

23. A method for treating cancer, comprising administering to a patient in need of treatment of such cancer, simultaneously or sequentially, a therapeutically effective amount of (1) tris(8-quinolinolato)gallium(III), or a pharmaceutically acceptable salt thereof, and (2) temozolomide.

24. The method of claim 23, wherein said cancer is melanoma or brain cancer.

25. A method for treating cancer, comprising administering to a patient in need of treatment of such cancer, simultaneously or sequentially, a therapeutically effective amount of (1) tris(8-quinolinolato)gallium(III), or a pharmaceutically acceptable salt thereof, and (2) lenalidomide or thalidomide.

26. The method of claim 25, wherein said cancer is multiple myeloma or MDS.

27. A method for treating cancer, comprising administering sequentially to a patient in need of treatment a therapeutically effective amount of (1) paclitaxel or Abraxane and (2) tris(8-quinolinolato)gallium(III), or a pharmaceutically acceptable salt thereof.

28. The method of claim 27, wherein said cancer is lung cancer.

29. A method for treating cancer, comprising administering sequentially to a patient in need of treatment a therapeutically effective amount of (1) tris(8-quinolinolato)gallium(III), or a pharmaceutically acceptable salt thereof, and (2) erlotinib.

30. The method of claim 29, wherein said cancer is lung cancer.
